(19) United States
(12) Patent Application Publication
(10) Pub. No.: US 2004/0219625 A1
(43) Pub. Date: Nov. 4, 2004

(54) Detection method for identifying hydrolases

(76) Inventors: Uwe T. Bornscheuer, Greifswald (DE); Markus Baumann, Lichtenwald (DE)

Correspondence Address: Morrison & Foerster, Suite 300, 1650 Tysons Boulevard, McLean, VA, 22102-3915, US

(21) Appl. No.: 10/477,895

(22) PCT Filed: May 21, 2002
(86) PCT No.: PCT/EP02/05570

(30) Foreign Application Priority Data
May 21, 2001 (DE) 101 24 799.0

(51) Int. Cl. C12Q 1/44

(52) U.S. Cl. 435/19

(57) ABSTRACT

The present invention relates to a detection or screening method for identifying hydrolases, based on an optical detection of liberated acetic acid, which is suitable in particular for high throughput screening (HTS) of prokaryotic or eukaryotic samples; to test kits for carrying out the method; to methods for isolating hydrolases with a modified profile of properties, such as, for example, with improved enantioselectivity, using this detection method; and to hydrolases isolated with the aid of this method.

The present invention relates to a detection or screening method for identifying hydrolases which is suitable in particular for high throughput screening (HTS) of prokaryotic or eukaryotic samples; to test kits for carrying out the methods; to methods for isolating hydrolases with a modified profile of properties, such as, for example, with improved enantioselectivity, using this detection method; and to hydrolases isolated with the aid of this method.

### PRIOR ART

Hydrolytic enzymes are versatile biocatalysts and are being increasingly used in organic synthesis [1]. Within this class of enzymes, lipases and esterases are frequently used because they convert a wide range of unnatural substrates, are usually very stable in organic solvents and show good to excellent stereoselectivity in the kinetic resolution of racemates or in the desymmetrization of prostereogenic compounds. Because of the enormous advances in the area of genetic engineering, the number of available biocatalysts is rapidly increasing. However, this makes it difficult to identify suitable enzymes with the aid of reactions on the laboratory scale followed by determination of the optical purity with the aid of gas chromatography or HPLC. In addition, the recently developed techniques for directed (molecular) evolution [2] require extremely exacting tests for high throughput screening (HTS) of large libraries, which usually contain 10⁴-10⁶ mutants.

Several test systems have been developed for rapid determination of the enantioselectivity E (also referred to as the enantiomer ratio) [3]. Kazlauskas et al. determine, for example, the initial rate of the hydrolase-catalyzed hydrolysis of p-nitrophenyl derivatives of pure chiral carboxylic acids (“quick E”) in the presence of resorufin tetradecanoate, to generate a competing reaction, in order to obtain more accurate values of E [4]. In a similar way, Reetz et al. use p-nitrophenyl derivatives in order to screen lipase mutants with improved enantioselectivity [5]. Problems arising from background absorption in the presence of culture supernatants and autohydrolysis of the p-nitrophenyl esters have been avoided by using a resorufin-based fluorescence assay for determining mutants of an esterase from Pseudomonas fluorescens with increased enantioselectivity for 3-phenylbutyric acid derivatives [6]. Another fluorogenic assay uses umbelliferone derivatives which, after hydrolase cleavage, are reacted with periodate and bovine serum albumin [7].

One disadvantage of all these test systems is the use of bulky chromophoric groups which usually differ considerably from the “true substrate”, that is to say acetate. There is accordingly a risk that the “wrong” mutants will be screened. In order to avoid this problem, Kazlauskas et al. have described a test in which both acetates of chiral alcohols and esters of chiral carboxylic acids are hydrolyzed in the presence of p-nitrophenol as pH indicator [8]. A stepwise test for screening for enantioselective hydrolases using bromothymol blue as pH indicator for detecting the activity, followed by gas chromatography to determine the enantioselectivity, has recently been described by the present inventors [9]. Another method, which is, however, not quantifiable, is based on the time-dependent IR-thermographic determination of the enantioselectivity [10]. A relatively accurate determination of the true values of E can be achieved by using “pseudo” enantiomers, with one form being isotope-labeled, such as, for example, deuterated acetate, followed by analysis by mass spectroscopy [11]. The particular disadvantages of this method are regarded as being the use of pure deuterated enantiomers and the high equipment costs. A “super HTS” has recently been described and entails determining the optical purity of FITC-labeled amines using a parallel capillary electrophoresis with chiral cyclodextrins [12]. This system is, however, confined to amines and likewise requires costly equipment.

### BRIEF DESCRIPTIONS OF THE INVENTION

It is an object of the present invention to provide a rapid, inexpensive method for detecting the activity of hydrolases such as, in particular, lipases and esterases, for determining enantioselectivity of hydrolases or for detecting hydrolases with an altered profile of properties. This method is intended to be suitable in particular for the HTS of microorganism libraries such as, for example, those produced by directed evolution.

We have found that this object is achieved by providing a test system which can be carried out rapidly and inexpensively in microtiter plates and is based on a coupled enzymatic conversion. The test system of the invention makes it possible in particular to determine hydrolase activity and enantioselectivity rapidly and reliably.

A first aspect of the invention relates to a detection or screening method for hydrolases such as, for example, enantioselective or stereoselective hydrolases, which comprises

a) incubating a reaction medium comprising as substrate at least one ester of acetic acid with an achiral, chiral or prochiral alcohol, with an analyte in which hydrolase activity is suspected, under acetic acid-liberating conditions; and

b) detecting the formation of acetic acid.

The substrate used for the detection, which is preferred according to the invention, of enantioselective or stereoselective hydrolases comprises at least one ester of acetic acid with a chiral or prochiral alcohol.

A further aspect of the invention relates to a test kit for carrying out the screening method of the invention, the test kit comprising besides conventional ingredients at least one chiral or prochiral substrate for the hydrolase.

Another aspect of the invention relates to a method for isolating natural artificial hydrolase mutants or hydrolase variants with an altered profile of properties, which comprises

a) preparing at least one analyte from a prokaryote or eukaryote;

b) investigating the analyte for hydrolase activity with the aid of a detection method of the invention;

c) in the event of a positive detection of hydrolase activity in the analyte, determining the profile of properties of the mutant or variant, and comparing with the profile of properties of a reference sample; and

d) if the profile of properties is detected to be altered by comparison with the reference sample, isolating the mutant or variant.

A final aspect of the invention relates to the use of the hydrolases which have improved enantioselectivity, and which have been isolated with the aid of a method of the invention, for preparing chiral esters and alcohols.

### DESCRIPTION OF THE FIGURES

The invention is described in the following sections with reference to the appended diagrams and figures. These show:

Diagram 1 die hydrolase-catalyzed reaction which liberates acetic acid. The latter is converted by adenosine 5′-triphosphate (ATP) and coenzyme A (CoA) into acetyl-CoA in the presence of acetyl-CoA synthetase (ACS). Citrate synthase (CS) catalyzes the reaction between acetyl-CoA and oxaloacetate to form citrate. The oxaloacetate necessary for this reaction is formed from N-malate and nicotinamide adenine dinucleotide (NAD+) in the presence of L-malate dehydrogenase (L-MDH). The initial rate of formation of acetic acid can thus be found from the increase in the extinction at 340 nm owing to the increase in the NADH concentration. If enantiopure acetates are used it is possible to determine the apparent enantioselectivity Eₐₚₚ;

Diagram 2 the model reaction for evaluating the coupled enzymatic acetate assay. Under the stated conditions, the esterase from Pseudomonas fluorescens shows high (R) selectivity (E=34);

Diagram 3 the acetates of secondary alcohols which were employed for evaluating the test. 1: alpha-phenylethyl acetate; 2: 2-acetoxy-3-butyne; 3:1-methoxy-2-propyl acetate; 4: 3-acetoxytetrahydrofuran; 5: pantolactone acetate;

FIG. 1 the approximately linear increase in the NADH concentration as a function of the time, observed during conversion of liberated acetic acid during the PFE-catalyzed hydrolysis of (R,S)-1. Autohydrolysis was not observed in the absence of the esterase;

FIG. 2 the relation between absorption at 340 nm and (A) amount of the substrate (R,S)-1 and (B) increasing esterase concentration;

FIG. 3 the result of the test system of the invention on hydrolysis of the acetates (R,S)-2 to 5 using lipase B from Candida antarctica (CAL-B);

FIG. 4 the initial rates determined using the optically pure. (R) and (S) enantiomers of (R,S)-1; the reactions were carried out using lyophilized PFE (A) or using culture supernatant containing PFE (B); it should be noted in this connection that FIG. 4B shows half the total activity.

### DETAILED DESCRIPTION OF THE INVENTION

The detection reaction of the invention can in principle be applied to known and previously undescribed, naturally occurring hydrolases or mutants and variants of such hydrolases. The natural hydrolases may in this connection be of eukaryotic, in particular plant or animal, or prokaryotic origin.

A hydrolase which has an “altered profile of properties” and which can be detected in a particularly simple manner with the aid of the method of the invention shows at least one of the following properties: altered, in particular increased, enzymic activity, altered, in particular increased, enantioselectivity, altered, in particular increased, temperature stability; and altered, in particular increased, stability in aqueous, organic and/or aqueous/organic liquids; in each case compared with a reference hydrolase with unaltered profile of properties, such as, for example, a naturally occurring hydrolase.

An “enantioselective hydrolase” within the meaning of the invention is a hydrolase which, in the hydrolyzing cleavage of a chiral or prochiral carboxylic ester, in particular of an ester of an achiral carboxylic acid, in particular acetic acid, and of a chiral or prochiral, for example secondary, alcohol, preferentially converts one enantiomer or one prostereogenic form. It should preferably be possible for at least one of the five reference substrates of the invention (diagram 3) to be converted enantioselectively.

The “screening method” of the invention encompasses both single measurements and series of measurements with in principle an unlimited number of single measurements.

The “analyte” of the invention encompasses all samples in which hydrolase activity is at least suspected. The analyte may be a hydrolase, dissolved in pure form or present in a mixture with other, for example cellular, material, and the other constituents should not suppress the hydrolase activity. The analyte may be, for example, a crude cell extract from prokaryotic cells, eukaryotic cells, organs or tissues or a culture supernatant from a eukaryotic or prokaryotic cell culture.

A “test kit” of the invention comprises in one or more separate parts the individual detection or test reagents and, where appropriate, test vessels therefor.

The test system of the invention for the rapid and reliable detection of hydrolase activity and, in particular, for the determination of its enantioselectivity via the formation of NADH in a coupled enzymatic conversion of acetic acid which is formed surprisingly meets the following essential criteria:

Stoichiometric relation between acetic acid formation by ester hydrolysis and NADH formation.

The hydrolase-catalyzed acetic acid liberation is the rate-determining step.

The increase in the extinction is linear over a large NADH concentration range.

The hydrolase has no effect on the subsequent conversion of acetic acid and on the components of the test kit (and vice versa).

The test makes it possible to determine accurately the enantioselectivity in order to be able to identify improved hydrolases.

The acetates are stable under slightly basic conditions (pH 8.1) during the reaction.

The “apparent” values of E (Eₐₚₚ) which are found from the ratio of the reaction rates of the pure enantiomers agree with the true values of E (Eₜᵣᵤₑ) as determined in competition experiments. Thus, for example, values for the apparent enantioselectivity Eₐₚₚ are obtained on use of optically pure (R) or (S) acetate of alpha-phenylethanol in separate wells of microtiter plates with recombinant esterase from Pseudomonas fluorescens (PFE). Lyophilized PFE from cell extracts gave a value of 30 for Eₐₚₚ. This roughly agrees with the values found for the hydrolysis of a racemic mixture (Eₜᵣᵤₑ=34), which was determined by gas chromatographic analysis.

The test can be applied to reactions in the presence of crude cell extract and culture supernatants although the value of E may in this case be found to deviate (for the above system Eₐₚₚ=21).

In the test system of the invention, initially acetate is liberated through the hydrolase-catalyzed hydrolysis of a chiral ester and is then determined by a cascade of enzymatic reactions [13].

In this screening method, in particular, the acetic acid is detected with the aid of a coupled enzymatic assay which is preferably an optical assay.

The acetic acid determination is preferably based on an inexpensive test kit which is already generally available and which was originally developed for food analysis. The test kit consists essentially of the enzymes acetyl-CoA, synthetase, citrate synthase and L-malate dehydrogenase. It additionally comprises the substrates and cosubstrates L-malate, ATP, NAD+, coenzyme A (CoA-SH), and buffers and stabilizers and, where appropriate, magnesium chloride. The acetate liberated in the hydrolytic reaction is converted stoichiometrically in this test system and brings about a change in the absorption (extinction) because of the reduction of NAD+ to NADH. This change can be determined by spectrophotometric measurement at 340 nm, 334 nm or 365 nm. The spectrophotometric measurement allows in particular the initial rate of the formation of NADH to be determined. The hydrolase activity can be determined on the basis of the stoichiometric relation between acetic acid liberation and NADH formation.

It is possible to use as analyte in the method of the invention the hydrolases as pure substances but also, in particular, crude cell extracts or a culture supernatant of a cultivated microorganism.

The overall detection reaction (mixing of analyte and enzymes and substrates for detection reaction and optical evaluation of the reaction mixture) preferably takes place in microtiter plates. In this case, the acetic acid formed by ester hydrolysis of the chiral ester employed (optically pure or racemate) is consumed with stoichiometric formation of NADH, and the NADH formation is determined optically at a suitable measurement wavelength, e.g. 340 nm, in each well of the microtiter plate.

In the coupled enzymatic assay of the invention, the stoichiometric formation of NADH takes place because acetic acid is converted enzymatically (in particular catalyzed by acetyl-CoA synthetase with consumption of ATP and coenzyme A) into acetyl-CoA which is converted stoichiometrically with oxaloacetate into citrate in an enzyme-catalyzed reaction with the aid of citrate synthase. The oxaloacetate necessary for this is formed enzymatically from L-malate in the presence of NAD+, with liberation of NADH, with the aid of L-malate dehydrogenase (see diagram 1). The reaction preferably takes place in buffered (pH about 7.5 to 8.5, in particular pH 8.1) medium in the presence of suitable other adjuncts such as magnesium chloride and, where appropriate, enzyme stabilizers.

The method of the invention is particularly applicable to hydrolases which are capable of cleaving carboxylic esters such as, for example, acetic esters. Such hydrolases are selected in particular from esterases (E.C.3.1.1.1), lipases (E.C.3.1.1.3), amidases (E.C.3.5.1.4), acylases (E.C.3.5.x.y) and proteases (E.C.3.4.x.y).

Examples of substrates which can be employed according to the invention are alpha-phenylethyl acetate, 2-acetoxy-3-butyne, 1-methoxy-2-propyl acetate, 3-acetoxy-tetrahydrofuran and pantolactone acetate, each in optically pure form or as mixture thereof, in particular racemate.

In a particularly preferred embodiment, the screening method of the invention is carried out in such a way that the hydrolase-catalyzed acetate formation is the rate-determining step of the overall detection method.

A particularly preferred area of application of the detection method of the invention is an HTS method for determining the hydrolase activity and/or selectivity in extracts or culture supernatants of natural or genetically modified microorganisms in which a large number of samples is investigated systematically for activity or improved enantioselectivity of a particular hydrolase.

A further particular configuration of the screening method consists of preceding the detection reaction by a cultivation of a large number of microorganisms to be analyzed in the wells of microtiter plates and preparing the analyte from the resulting microorganism cultures.

The screening method of the invention is suitable not only for determining the enzymic activity of the hydrolase in the analyte, but also for determining the enantioselectivity for the hydrolase in the analyte.

A specific embodiment of the method of the invention for isolating natural or artificial hydrolase mutants or hydrolase variants with increased enantioselectivity comprises

a) cultivating a microorganism, in particular a recombinant microorganism, which expresses the coding sequence for a hydrolase which has been generated by in vitro mutagenesis, in particular by saturation mutagenesis or targeted (“directed”) evolution and which possibly expresses hydrolase activity;

b) preparing at least one analyte from the culture;

c) investigating the analyte(s) with the aid of a screening method of the invention for hydrolase activity;

d) in the event of a positive detection of hydrolase activity in the analyte, determining the enantioselectivity and comparing the value found with the value for a reference sample; and

e) if the enantioselectivity is detected as increased by comparison with the reference sample, the enzyme is isolated from the culture, where appropriate, after further cultivation of the microorganism.

It is possible in an analogous manner to detect and isolate hydrolases with improved activity, stability to temperature and/or organic solvents and/or improved stereoselectivity.

Evaluation of the Test System:

In order to verify whether the test system of the invention meets the above criteria, the hydrolysis of (R,S)-alpha-phenylethyl acetate (also referred to as (R,S)-1) by a recombinant esterase from Pseudomonas fluorescens (PFE) was used as model reaction (diagram 2). Earlier results had shown that PFE converts this substrate with high enantioselectivity in the hydrolysis and in acylation reactions in organic solvents [14].

The first hydrolysis reactions were carried out on the ml scale using lyophilized PFE (1 mg/ml), 50 mM (R,S-1) and the acetic acid test kit described below. In addition, the conversion and the enantiomeric excess were determined by gas chromatographic analyses of samples from the reaction mixture (diagram 2). Incubation of (R,S)-1 at pH 8.1 in the presence of the test kit but in the absence of PFE led to no NADH formation. It is thus possible to preclude autohydrolysis of (R,S)-1. It was additionally found that neither the activity nor the enantioselectivity of PFE was influenced by the components of the test kit (results of control experiments which are not shown). The maximum observed increase in the extinction at 340 nm was 0.3 per minute. This appears to be the maximum reaction rate with the coupled test system of the invention. However, a lower rate is advisable for greater accuracy.

Transfer of the Test System to Microtiter Plates:

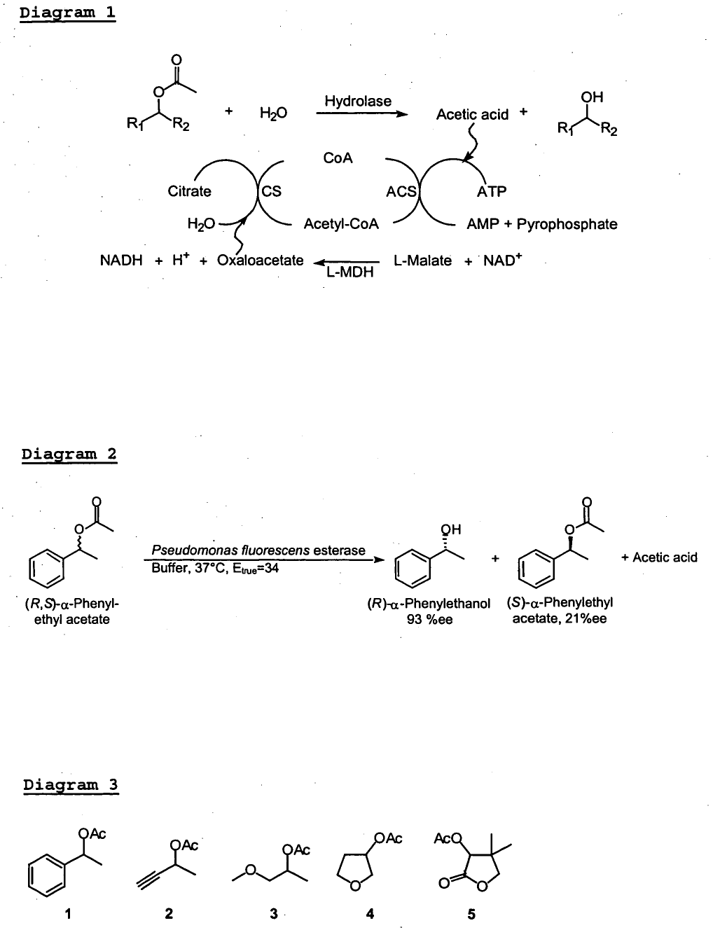

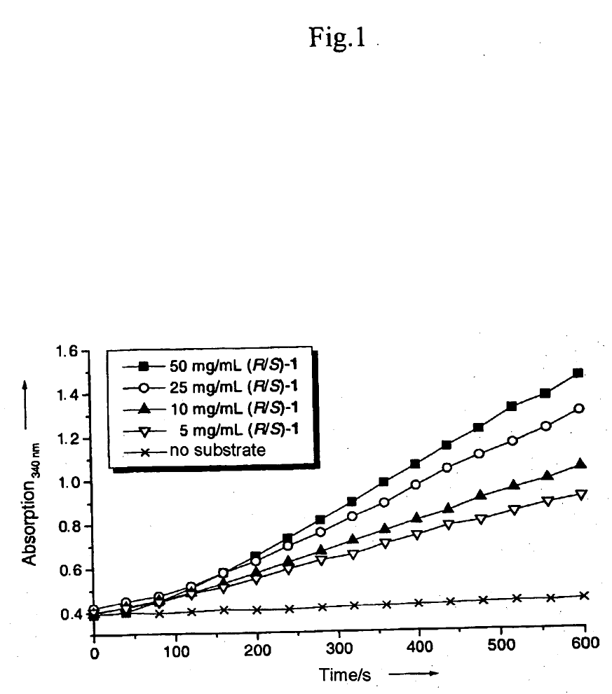

The reaction in microtiter plates (MTP) showed an approximately linear increase in the extinction at 340 nm over a period of 10 minutes on use of a PFE concentration of 2 mg/ml. The (R,S)-1 concentration was varied in the range 5-50 mg/ml (see FIG. 1).

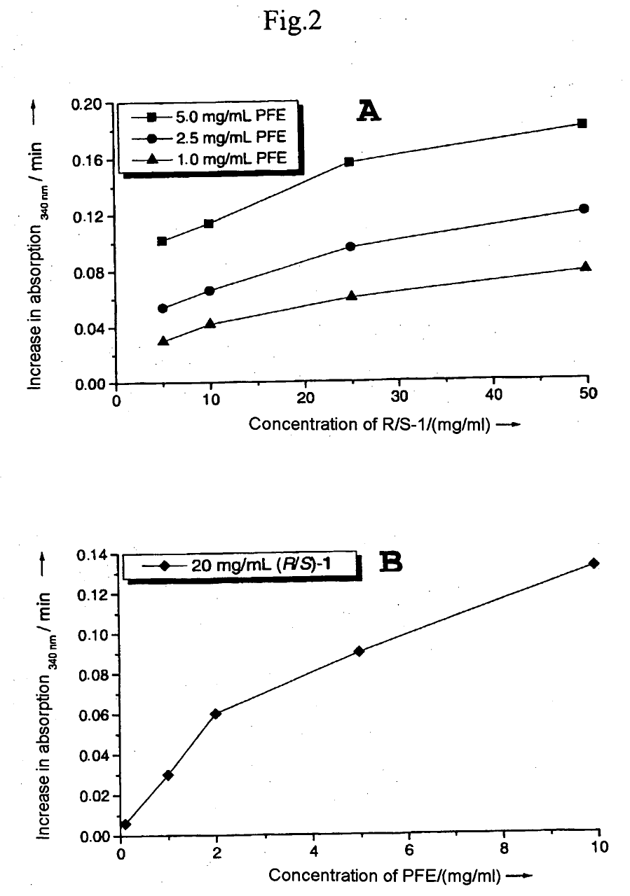

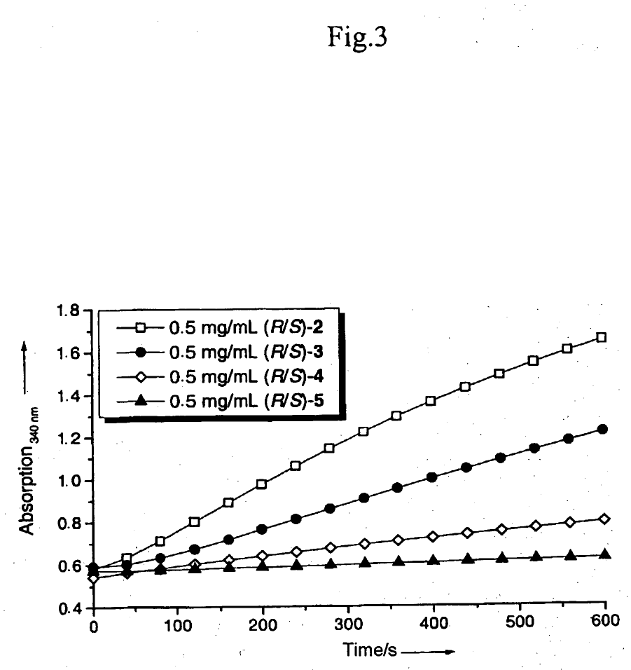

In contrast to this, the increase in extinction per minute at a measurement wavelength of 340 nm shows a less linear behavior on variation of the PFE concentrations or of the (R,S)-1 concentration (FIGS. 2A and B). However, FIG. 2B shows that a reliable detection of activity is possible even below or above a concentration of 2 mg/ml PFE.

Even at concentrations of 10 mg/ml PFE and 20 mg/ml (R,S)-1, the enzyme-coupled detection cascade with liberation of NADH is still twice as fast as the esterase reaction.

Tests with lower hydrolase and substrate concentrations are, however, preferred because they make more accurate determination of the reaction rate possible.

Utilizability of Other Substrates and Enzymes:

Besides the model compound 1, the test can likewise be utilized for chiral esters of other secondary alcohols (see diagram 3). Lipase B from Candida antarctica (CAL-B) was used as catalyst for these compounds [15]. The coupled conversion of the (R,S) acetates of these compounds show decreasing reaction rates in the following sequence: 2-acetoxy-3-butyne 2>1-methoxy-2-propyl acetate 3>3-acetoxy-tetrahydrofuran 4>pantolactone acetate 5. This observation agrees with earlier results [9] and demonstrates that the test system of the invention can also be applied to determining the activity of other hydrolases.

Enantioselectivity Determination:

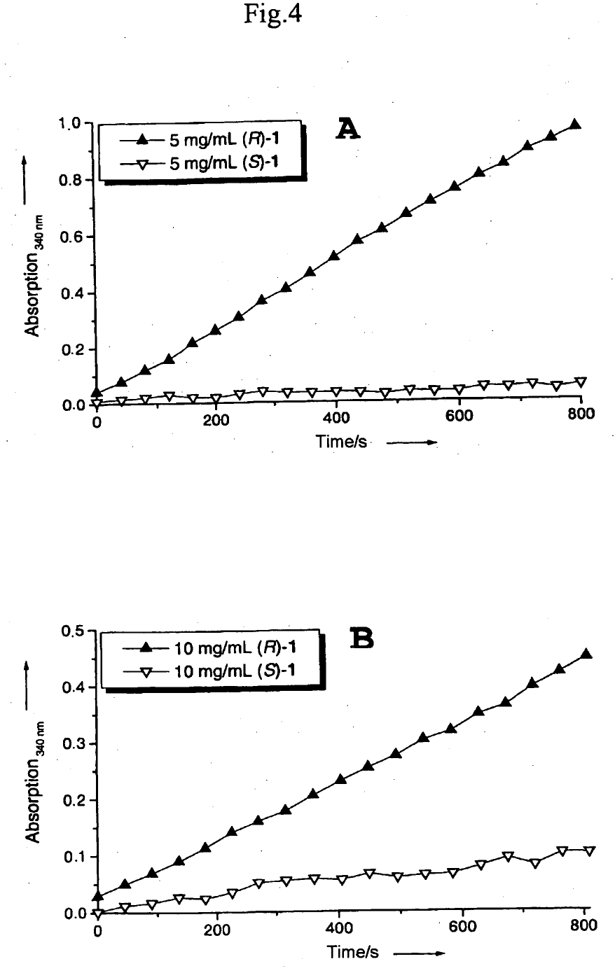

Measurement of the initial rates of acetic acid liberation from the pure enantiomers (R)-1 or (S)-1 (5 mg/ml) with an esterase concentration of 1.0 mg/ml PFE (lyophilized crude extract) showed almost exclusively an increase in the extinction for the (R) enantiomer. This illustrates the high enantioselectivity of PFE (FIG. 4A). The apparent enantioselectivity Eₐₚₚ was 30. This roughly corresponds to a value of E=34 shown in diagram 2. In a further approach for a PFE-containing culture supernatant was divided into two parts in order to ensure that the same enzyme concentration is used for both enantiomers. Measurement of the initial rate for the two enantiomers showed that impurities from the cultivation, such as cell fragments or media components, influence the quality of the determination of the liberated acetic acid (FIG. 4B). It cannot be ruled out that acetic acid from the cultivation also contributes to this observation. The influences associated with the use of crude culture supernatant are also reflected by the reduced initial rate and the lower enantioselectivity (Eₐₚₚ=21).

On the other hand, it should be noted that expression of PFE is possible in a controlled manner by cultivation of recombinant E. coli. It is thus possible to predict relatively accurately the amount of PFE per well. The lower accuracy of the test system on use of crude culture supernatant might be improved, for example, by interpolating a purification step, for example using a microtiter plate on which expressed PFE is purified by metal ion affinity chromatography based on a cloned-in histidine tag.

Experimental Part

Materials

The racemic acetates 1-3 were acquired in the highest state of purity by purchase. Optically pure (R)-1 and (S)-1, and the acetates (R,S)-4-5 were prepared from the corresponding alcohols with acetyl chloride in pyridine using standard methods. Lipase B from Candida antarctica (CHIRAZYME® L-2, CAL-B) was purchased from Roche Molecular Biochemicals (Penzberg, Germany). The recombinant esterase from Pseudomonas fluorescens (PFE) was prepared in a known manner [16].

The test kit for determining acetic acid (originally produced and marketed by Roche Diagnostics, Penzberg, Germany) was purchased from R-Biopharm GmbH (Darmstadt, Germany) and used in accordance with the manufacturer's instructions.

The spectrophotometric determination of the NADH concentration was carried out at 340 nm on the ml scale in an Ultrospec 3000 photometer (Pharmacia Biotech Ltd., Uppsala, Sweden) and on the μl scale using a fluorimeter (FLUOstar, BMG Lab Technologies, Offenburg, Germany).

Calculations

The enantiomeric excess (% ee) and the values of Eₜᵣᵤₑ were determined as described by Chen et al. [3] by gas chromatographic analysis using a heptakis(2,3,6-tri-O-methyl)-β-cyclodextrin column (CS Chromatographie Service GmbH, Langerwehe, Germany) in a Fisons Instrument HRGC Mega 2 type gas chromatograph (ThermoQuest Analytische Systeme GmbH, Egelsbach, Germany). The apparent values of E (Eₐₚₚ) were determined from the ratio of the initial rates of NADH formation determined in each case for the (R) and the (S) enantiomer using the data found with the above test kit.

Calculation of the enantiomeric excess (% ee):

%ee=(A−B)/(A+B)*100,

with A, B=mole fractions of the respective enantiomers and A>B

Calculation of the E value:

E=[In(1−c)*(1−eeₛ)]/[In(1−c)*(1+eeₛ)]

with c=conversion, eeₛ=enantiomeric excess of the substrates alternatively when the enantiomeric excess of the product (eeₚ) is known:

E=[In(1−c*(1+eeₚ)]/[In(1−c*(1−eeₚ)]

General Method for Determining Acetic Acid in a Microtiter Plate Test

The test kit (150 μl) was mixed with PFE (20 μl, 2 mg/ml, unless stated otherwise) or with CAL-B (2 mg/ml) in solution. The reactions were started by adding a solution of substrates 1-5 (20 μl, with the concentrations stated in the figures) in sodium phosphate buffer (10 mM, pH 7.3). Mixtures of the test kit with buffer or cell lysate from uninduced E. coli DH5α which comprises the gene for recombinant esterase from Pseudomonas fluorescens served as control. The reaction rates were determined using optically pure substrates in a similar way.

Esterase-catalyzed Hydrolysis of α-phenylethyl Acetate (1)

PFE (1 mg/ml) and (R,S)-1 (50 mM) were added to sodium phosphate buffer (500 μl, 10 mM, pH 7.3) in a reaction vessel. The reaction mixture was shaken at 37° C. for one hour and then extracted with diethyl ether (500 μl). The organic phase was removed, dried and used directly for GC analysis to determine the conversion and the enantioselectivity. In addition, reactions were carried out in the presence of test kit components in order to establish the effect of the slightly basic pH (8.1) on the esterase performance.

Expression of Pseudomonas fluorescens Esterase in Microtiter Plates:

Luria Bertani medium (200 μl) containing ampicillin (0.1 mg/ml) in each well were inoculated with E. coli DH5α which contained the plasmid pJOE2792.1 which codes for the Pseudomonas fluorescens esterase gene [16]. The microtiter plates were shaken at 37° C. (190 rpm) in a Multitron HT shaker (Infors AG, Bottmingen, Switzerland) until the optical density at 578 nm reached 0.3. Expression of the esterase was induced by adding L-rhamnose solution (20 μl, 2%, w/v). After 16 hours, the cells were spun down (10 minutes at 4000 rpm and 4° C.)(5810 R centrifuge, Eppendorf-Netheler-Hinz GmbH, Hamburg, Germany). The cells were resuspended in sodium phosphate buffer (150 μl, 10 mM, pH 7.3) and disrupted in two freeze/thaw cycles. Finally, cell fragments were spun down and the supernatants were stored in ice or diluted with sodium phosphate buffer before use thereof.

The test described herein is the first test system for which it has been possible to show utilizability for a direct determination of hydrolase in enantioselectivity in a crude cell extract. It has the advantage that the test principle can be employed directly for HTS in microorganism libraries like those obtained by directed evolution. The analysis time for each test determination of the enantioselectivity is about 3-4 minutes (plus about 1-2 minutes required for the pipetting steps). It is thus possible to analyze in a microtiter plate with 96 wells about 45 enzymes (2 wells for each enzyme, 6 wells for control mixtures). In this way, 540 values of E are obtained per hour. It is thus possible to screen about 13 000 mutants each day. In addition, the test of the invention is relatively low-cost because one acetic acid test kit is sufficient for about 660 reactions. The method of the invention is not restricted to the esterases and lipases described by way of example and to the determination of their enantioselectivity. Other enzymic activities which bring about liberation of acetic acid, such as protease-catalyzed or amidase-catalyzed hydrolyses of amides, ought likewise to be analyzable in accordance with the principle of the invention.

### LIST OF REFERENCES

[1] a) R. N. Patel, Stereoselective Biocatalysis, Marcel Dekker, New York, 2000; b) K. Faber, Biotransformations in Organic Chemistry, Springer, Berlin, 2000; c) U. T. Bomscheuer, R. J. Kazlauskas, Hydrolases in organic synthesis—regio—and stereoselective biotransformations, Wiley-VCH, Weinheim, 1999.

[2] a) U. T. Bornscheuer, M. Pohl, Curr. Opin. Chem. Biol. 2001, 5,137-143; b) U. T. Bornscheuer, Biocat. Biotransf. 2001, in press; c) U. T. Bornscheuer, Angew. Chem. 1998, 110, 3285-3288; Angew. Chem. Int. Ed. Engl. 1998, 37, 3105-3108; d) F. H. Arnold, A. A. Volkov, Curr. Opin. Chem. Biol. 1999, 3, 54-59; e) J. D. Sutherland, Curr. Opin. Chem. Biol. 2000, 4, 263-269; f) M. B. Tobin, C. Gustafsson, G. W. Huisman, Curr. Opin. Struct. Biol. 2000, 10, 421-427.

[3] C. S. Chen, Y. Fujimoto, G. Girdaukas, C. J. Sih, J. Am. Chem. Soc. 1982, 104, 7294-7299.

[4] L. E. Janes, R. J. Kazlauskas, J. Org. Chem. 1997, 62, 4560-4561.

[5] M. T. Reetz, A. Zonta, K. Schimossek, K. Liebeton, K.-E. Jaeger, Angew. Chem. 1997, 109, 2961-2963; Angew. Chem. Int. Ed. Engl. 1997, 36, 2830-2832.

[6] E. Henke, U. T. Bornscheuer, Biol. Chem. 1999, 380,1029-1033.

[7] F. Badalassi, D. Wahler, G. Klein, P. Crotti, J.-L. Reymond, Angew. Chem. 2000, 112, 4233-4236; Angew. Chem. Int. Ed. Engl. 2000, 39, 4067-4070.

[8] a) L. E. Janes, A. C. Löwendahl, R. J. Kazlauskas, Chem. Eur. J. 1998,4,2324-2331; b) A. M. F. Liu, N. A. Somers, R. J. Kazlauskas, T. S. Brush, F. Zocher, M. M. Enzelberger, U. T. Bornscheuer, G. P. Horsman, A. Mezzetti, C. Schmidt-Dannert, R. D. Schmid, Tetrahedron: Asymmetry 2001, 12, 545-556

[9] M. Baumann, B. Hauer, U. T. Bornscheuer, Tetrahedron: Asymmetry 2000, 11, 4781-4790.

[10] M. T. Reetz, M. H. Becker, K. M. Kühling, A. Holzwarth, Angew. Chem. 1998, 111, 2792-2795; Angew. Chem. Int. Ed. Engl. 1998, 37, 2647-2650.

[11] M. T. Reetz, M. H. Becker, H. W. Klein, D. Stöckigt, Angew. Chem. 1999, 111, 1872-1875; Angew. Chem. Int. Ed. Engl. 1999, 38, 1758-1761.

[12] M. T. Reetz, K. M. Kühling, A. Deege, H. Hinrichs, D. Belder, Angew. Chem. 2000, 112, 4049-4052; Angew. Chem. Int. Ed. Engl. 2000, 39, 3891-3893.

[13] a) H. U. Bergmeyer (Ed.), Methods of Enzymatic Analysis, Vol. 1, Wiley-VCH, Weinheim 1974, p. 112-117; b), H. U. Bergmeyer (Ed.), Methods of Enzymatic Analysis, Vol. 3, Wiley-VCH, Weinheim 1974, p. 1520-1528.

[14] N. Krebsfänger, K. Schierholz, U. T. Bornscheuer, J. Biotechnol. 60 (1998) 105-111.

[15] E. M. Anderson, K. M. Larsson, O. Kirk, Biocatal. Biotransform. 1998, 16, 181-204.

[16] N. Krebsfänger, F. Zocher, J. Altenbuchner, U. T. Bornscheuer, Enzyme Microb. Technol. 21 (1998) 641-646.

1. A method for determining enantioselectivity or stereoselectivity of a hydrolase, which comprises:
incubating under acetic acid-liberating conditions a reaction medium comprising as a substrate at least one ester of acetic acid with an achiral, chiral or prochiral alcohol, and an analyte in which hydrolase activity is suspected; and
determining formation of NADH wherein a stoichiometric amount of acetic acid, is consumed during the incubating step.

2. A method as claimed in claim 1, wherein determining the formation of NADH is performed in an optical assay.

3. A method as claimed in claim 1, wherein the analyte is a crude cell extract of a culture supernatant of a cultivated microorganism.

4. A method as claimed in claim 1, wherein determining the formation of NADH is performed in a microtiter plate.

5. A method as claimed in claim 1, wherein the stoichiometric formation of NADH takes place in a coupled enzymatic assay through enzymatic conversion of acetic acid into acetyl-CoA which is converted stoichiometrically in an enzyme-catalyzed reaction with oxaloacetate into citrate, with oxaloacetate being formed enzymatically from L-malate in the presence of NAD+ with liberation of NADH.

6. A method as claimed in claim 1, wherein NADH is detected spectrophotometrically via extinction at 340 nm.

7. A method as claimed in claim 1, wherein the hydrolase is selected from the group of enzymes consisting of esterases, lipases, amidases, acylases and combinations thereof.

8. A method as claimed in claim 1, wherein the substrate is in optically pure form.

9. A method as claimed in claim 1, wherein the substrate is selected from the group of substrates consisting of achiral, chiral and prochiral forms of alpha-phenylethyl acetate, 2-acetoxy-3-butyne, 1-methoxy-2-propyl acetate, 3-acetoxytetrahydrofuran, pantolactone acetate and combinations thereof.

10. A method as claimed in claim 1, wherein formation of acetic acid is rate-determining.

11. A method as claimed in claim 1, which is a high throughput screening method.

12. A method as claimed in claim 1, wherein the determining step is preceded by cultivation of a microorganism and preparation of the analyte from the microorganism culture.

13. A method as claimed in claim 11 which is capable of determining the hydrolase activity or selectivity in extracts or culture supernatants of natural or genetically modified microorganisms.

14. A method for isolating a variant microorganism containing a natural or artificial hydrolase, which comprises:
preparing an analyte from the variant microorganism;
investigating the analyte for hydrolase activity according to the method as claimed in claim 1;
determining a profile of properties of the variant microorganism whose analyte is positive for hydrolase activity, and comparing the profile of properties with at least a similar profile for a reference microorganism; and
isolating the variant microorganism.

15. A method as claimed in claim 14, wherein the microorganism is a recombinant microorganism which expresses a coding sequence for a hydrolase.

16. A method as claimed in claim 14, wherein the profile of properties comprises at least one of the properties selected from the group of properties consisting of: enzymic activity, enantioselectivity, temperature stability; stability in aqueous, organic or aqueous/organic liquids, and combinations thereof.

17. A method as claimed in claim 14, wherein the variant microorganism is a natural or recombinant mutant microorganism.

18-20. (Canceled)

21. A method as claimed in claim 1, wherein the analyte is a cultivated plant or animal cells.

22. A method as claimed in claim 1, wherein the analyte is derived from plants, animals or organs or parts thereof.

23. A method as claimed in claim 1, wherein the substrate is a racemic mixture of substrates.

24. A method as claimed in claim 12, wherein the microorganism is cultured in a microtiter plate.

25. A method as claimed in claim 14, wherein the variant microorganism is prokaryotic.

26. A method as claimed in claim 14, wherein the variant microorganism is eukaryotic.

27. A method as claimed in claim 14, wherein the hydrolase is generated by in vitro mutagenesis.

28. A method as claimed in claim 14 wherein the hydrolase is generated by saturation mutagenesis.

29. A method as claimed in claim 14 wherein the hydrolase is generated by directed evolution.

30. A method for preparing a chiral ester with the variant microorganism of claim 14.

31. A method for preparing a chiral alcohol with the variant microorganism of claim 14.

32. A kit for identifying a hydrolase comprising:
a reaction medium containing as a substrate at least one ester of acetic acid with an alcohol, and an analyte in which hydrolase activity is suspected, wherein the reaction medium can be incubated under acetic acid liberating conditions;
asay for detecting NADH from a stoichiometric amount of acetic acid consumed during incubating.

33. A kit as described in claim 32, wherein the substrate is a chiral or prochiral substrate for said hydrolase.

34. A kit as described in claim 32 further comprising one or more additional constituents.

35. A kit as described in claim 34, wherein the one or more additional constituants are selected from the group consisting of magnesium chloride, enzyme stabilizers, and buffered medium.

36. A kit as described in claim 32, wherein the alcohol is achiral, chiral or prochiral.

37. A method for preparing a chiral ester or alcohol comprising:
isolating a variant microorganism containing a natural or artificial hydrolase comprising the steps of:
preparing an analyte from the variant microorganism;
incubating under acetic acid-liberating conditions a reaction medium containing said analyte and, as a substrate, at least one ester of acetic acid with an alcohol; and
determining formation of NADH from a stoichiometric amount of acetic acid consumed during incubating; and
preparing the chiral ester or alcohol from the variant microorganism.

38. A method as described in claim 37, wherein the alcohol is chiral.

39. A method as described in claim 37, wherein the alcohol is prochiral.

40. A method as described in claim 37, wherein the alcohol is achiral.
